(19) United States
(12) Patent Application Publication
(10) Pub. No.: US 2003/0027234 A1
(43) Pub. Date: Feb. 6, 2003

(54) Methods for detecting Down's syndrome

(76) Inventors: Murugan R. Pandian, Mission Viejo, CA (US); Julie Y. Lu, Mission Viejo, CA (US)

Correspondence Address: Donald E. Stout, Stout, Uxa, Buyan & Mullins, LLP, Suite 300, 4 Venture, Irvine, CA, 92618, US

(21) Appl. No.: 09/918,317

(22) Filed: Jul. 30, 2001

(51) Int. Cl. G01N 33/53

(52) U.S. Cl. 435/7.93

(57) ABSTRACT

Methods for detecting Down's syndrome in a fetus of a pregnant woman include screening serum samples obtained from the pregnant woman for abnormal levels of invasive trophoblast antigen. In particular, serum levels of invasive trophoblast antigen are compared to a standard. The methods can also be practiced using at least one additional marker.

### SUMMARY OF THE INVENTION

The present invention provides methods for detecting Down's syndrome in a fetus of a pregnant woman. In particular, the methods comprise screening biological samples for abnormal levels of invasive trophoblast antigen (ITA).

In one embodiment of the invention, a method for detecting Down's syndrome in a fetus of a pregnant woman comprises the steps of: a) contacting a serum sample from a pregnant woman in a chemilumninescent assay with an antibody that specifically binds to ITA; and b) comparing the amount of ITA in the sample to a standard ITA amount obtained from a population of pregnant women with normal fetuses, wherein a higher amount of ITA in the sample as compared to the standard ITA amount indicates the presence of Down's syndrome in the fetus.

In another embodiment of the invention, the method comprises the steps of contacting a biological sample from a pregnant woman for ITA, and comparing the amount of the ITA in the sample to a standard value. The sample may be obtained from the pregnant woman during the first trimester or second trimester of the pregnancy. In certain embodiments of the invention, the sample is either a whole blood sample, a serum sample, or a urine sample. In one embodiment of the invention, the sample is screened for ITA using an antibody, preferably a monoclonal antibody (mAb), that specifically binds ITA. The monoclonal antibody is able to specifically bind ITA obtained from whole blood or serum samples. One example of a monoclonal antibody is a mAb raised against the human chorionic gonadotropin (hCG) beta subunit. One example is designated B207, as described herein. Another monoclonal antibody is a mAb that specifically binds ITA. One example is designated B152.

In other embodiments of the invention, the foregoing method may be practiced by screening the sample for one or more additional biological markers associated with pregnancy. Examples of markers include, but are not limited to, hCG, beta-subunit hCG, beta-core hCG, unconjugated estriol (UE3), alpha-fetoprotein (AFP), leptin, prorenin, renin, DHEA-S, leukocyte acid phosphatase, inhibin, pregnancy associated plasma protein A (PAPP-A), AFP-L3, P43, superoxide dismutase (SOD), proMBP, fetal DNA, insulin-like growth factor binding proteins 3 (IGFBP3), CA 125, placental lactogen, Hp2FF, serum sialytransferase, s100b protein, schwangers chafts protein 1 (SPI), activin A/follistatin, fetal antigen (FA-2) and placental alkaline phosphatase (PALP). Ultrasound screens may also be practiced in the methods of the invention.

The methods of the invention may be practiced using a sandwich immunoassay, wherein the B152 mAb is the capture antibody, and the B207 mAb is the detection antibody. In one embodiment of the invention, the sandwich immunoassay is a chemiluminescent immunoassay. In another embodiment, the B152 capture antibody is coupled to biotin, which couples with streptavidin-coated magnetic particles, and the B207 detection antibody is coupled to an acridinium ester.

The screening steps of the foregoing methods may be automated. The foregoing methods provide high detection rates of Down's syndrome. For example, and not by way of limitation, the methods result in detection rates greater than about 80%, and preferably greater than about 85%, and even more preferably greater than about 90%. In a further embodiment of the invention, the methods disclosed herein result in detection rates of Down's syndrome greater than about 95%.

An increased risk that a fetus has Down's syndrome may be determined if the ITA amount in a biological sample is greater than about the 50^{th} percentile of the ITA concentration found in a population of women with normal pregnancies. In another embodiment, the ITA amount in the sample may be greater than the 95^{th} percentile of the ITA concentration found in a population of women with normal pregnancies.

Any feature or combination of features described herein are included within the scope of the present invention provided that the features included in any such combination are not mutually inconsistent as will be apparent from the context, this specification, and the knowledge of one of ordinary skill in the art.

Additional advantages and aspects of the present invention are apparent in the following detailed description and claims.

### BRIEF DESCRIPTION OF THE FIGURES

FIG. 1 illustrates N-linked and O-linked oligosaccharides on ITA found in Down's syndrome. FIG. 1A shows the N-linked hyperglycosylated β-subunit. FIG. 1B shows the O-linked hyperglycosylated β-subunit. Abbreviations are as follows: SA for sialic acid; Gal for galactose; GlcNAc for N-acetylglucosamine; Man for manose; and Fuc for fucose.

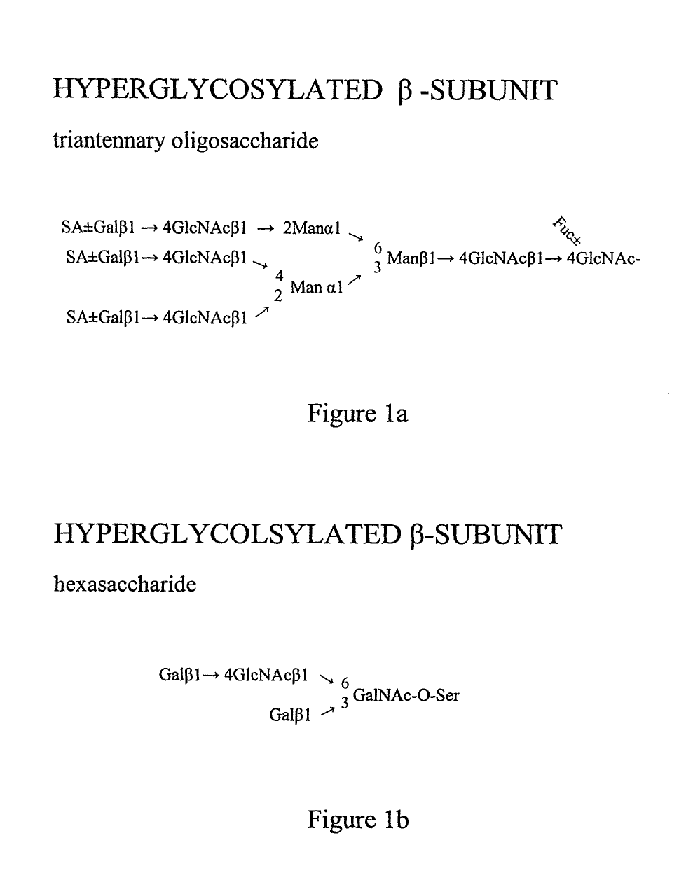

FIG. 2 is a graph of serum ITA concentration (ng/mL) versus gestational age (weeks). Open circles (∘) represent ITA values in serum from pregnant women who had Down's syndrome fetuses. Solid circles (●) represent ITA values in serum from pregnant women who had normal fetuses.

FIG. 3 is a graph of serum ITA concentration (ng/mL) versus gestational age (weeks). The data represent ITA concentrations in serum from pregnant women who had normal fetuses. The data represent the median values of ITA concentration.

FIG. 4 is a graph of ITA concentration (ng/mL) versus gestational age (weeks). Open circles (∘) represent ITA values from serum of pregnant women who had Down's syndrome fetuses. Asterisks (*) represent ITA values from serum of pregnant women who had fetuses with Down's syndrome. The serum from these women, in particular, had been frozen and thawed more than once, whereas the serum from the other women (∘) had not been frozen and thawed. The open squares (□) represent ITA values from serum of pregnant women who had normal fetuses. The 50^{th} and 95^{th} percentiles of ITA concentration for normal pregnancies are illustrated as a log Gaussian line fitted for the normal pregnancy values.

FIG. 5 is a graph of serum ITA values expressed as Multiples of Medians (MoM) from serum samples obtained from pregnant women who had normal fetuses (curve on the left), and from samples obtained from pregnant women who had fetuses with Down's syndrome (curve on the right).

FIG. 6 is a graph of ITA, measured as the “Multiple of Medians” (MoM) versus gestational age (weeks). Open circles (∘) represent ITA values from serum of pregnant women who had Down's syndrome fetuses. Asterisks (*) represent ITA values from serum of pregnant women who had fetuses with Down's syndrome. The serum from these women, in particular, had been frozen and thawed more than once, whereas the serum from the other women (∘) had not been frozen and thawed. The open squares (□) represent ITA values from serum of pregnant women who had normal fetuses. The 95^{th} percentile of ITA (MoM) is indicated as a log Gaussian line.

FIG. 7 is a graph of ITA values expressed as Multiples of Medians (MoM) versus percentile. Solid squares (▪) represent ITA values from serum samples of pregnant women who had Down's syndrome fetuses. Open squares (□) represent ITA values from serum samples of pregnant women who had normal fetuses.

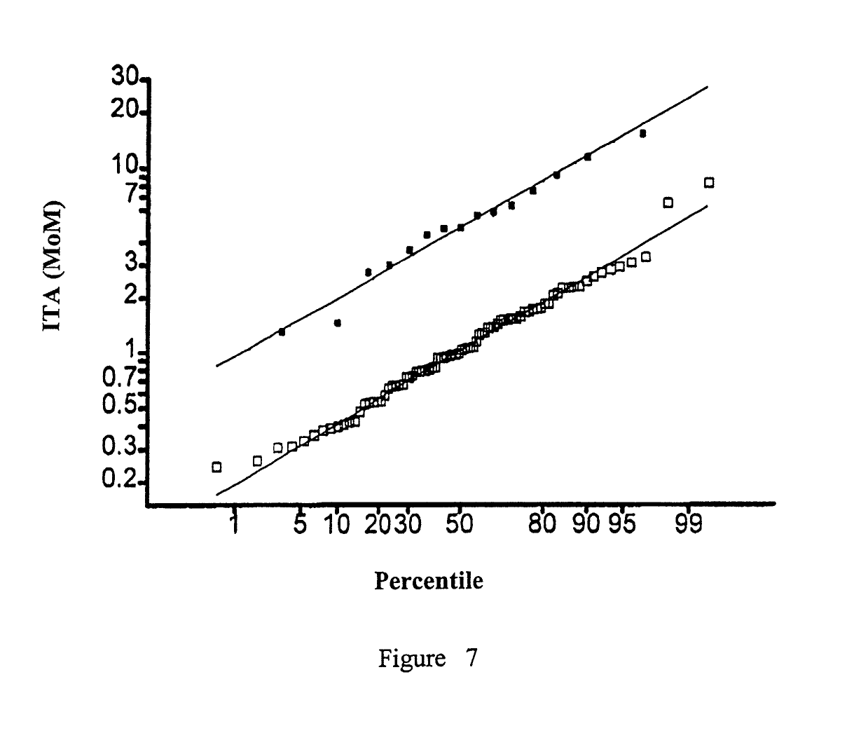

### DETAILED DESCRIPTION OF THE INVENTION

Unless defined otherwise, all technical and scientific terms used herein have the same meaning as commonly understood by those of ordinary skill in the art to which this invention belongs. For purposes of the present invention, the following terms are defmed below.

As used herein, “invasive trophoblast antigen” (ITA) is a glycoprotein hormone secreted by trophoblast cells of the placenta of pregnant women. ITA is also called hyperglycosylated hCG (h-hCG). ITA is similar to C5 hCG, which is a nicked h-hCG obtained from a choriocarcinoma patient. ITA, as defined, also includes fragments of ITA, or variants of ITA. In particular, ITA encompasses molecules that exhibit similar biological activities or expression patterns to ITA and that exhibit aberrant carbohydrate levels as compared to normal hCG including, nicked hCG, α-subunits of hCG, β-subunits of hCG, or any combination thereof. Examples of ITA isoforms include isoforms that comprise 57% triantennary N-linked oligosaccharides and 68% hexasaccharide-type O-linked oligosaccharides. Another ITA isoform may comprise 48% triantennary N-linked oligosaccharides and 100% hexasaccharide-type O-linked oligosaccharides. In normal pregnancies, a relatively small proportion of more complex triantennary N-linked oligosaccharides (0-30%) and larger hexasaccharide-type O-linked sugar units (0-20%) are also found.

In one embodiment of the invention, ITA comprises fragments of ITA. For example, greater nicking is observed in ITA preparations compared to hCG preparations. For example, ITA may be nicked or cleaved at similar sites on its beta subunit, and dissociate to form a free alpha subunit and a nicked free hyperglycosylated beta-subunit. Nicked free beta-subunit of ITA can be further degraded to a beta-subunit core fragment comprising short disulfide-linked peptides, with traces of hyperglycosylation sugar moieties. The detection of the fragments of ITA to detect a fetus with Down's syndrome is within the scope of this invention.

As used herein, “normal pregnancy” is defined as a pregnancy wherein the cells of a fetus do not have an excessive amount of chromosomal material, for example, the cells of the fetus do not have an extra copy, or extra part, of chromosome 21.

As used herein, “antibody” refers to a polypeptide substantially encoded by an immunoglobulin gene or immunoglobulin genes, or fragments thereof, which specifically recognize and bind an antigen. The recognized immunoglobulin genes include the kappa, lambda, alpha, gamma, delta, epsilon, and mu constant region genes, as well as the immunoglobulin variable region genes. Antibodies include fragments, such as Fab′, F(ab)₂, Fabc, and Fv fragments. The term “antibody,” as used herein, also includes antibody fragments either produced by the modification of whole antibodies or those synthesized de novo using recombinant DNA methodologies, and further includes “humanized” antibodies made by now conventional techniques.

An antibody “specifically binds to” or “is immunoreactive with” a protein when the antibody functions in a binding reaction with the protein. In order for the antibody to bind to a protein, the protein and the antibody should contact. Accordingly, contacting a sample suspected of containing an antigen of interest with an antibody to the antigen will permit the antibody to specifically bind the antigen. The binding of the antibody to the protein permits determination of the presence of the protein in a sample in the presence of a heterogeneous population of proteins and other agents. Thus, under designated immunoassay conditions, the specified antibodies bind preferentially to a particular protein and do not significantly bind to other proteins present in the sample. Specific binding to a protein under such conditions requires an antibody that is selected for specificity for a particular protein. Several methods for determining whether or not a peptide is immunoreactive with an antibody are known in the art.

As used herein, a “capture antibody” is defined as an antibody, preferably a monoclonal antibody, attached to a substrate, preferably a solid substrate. The capture antibody is selected to specifically bind a particular, distinct epitope of ITA.

As disclosed herein, one capture antibody is designated B152, and may be attached to a solid substrate comprising magnetic particles. Monoclonal antibody B152 specifically binds ITA. The hybridoma producing the B152 monoclonal antibody was deposited on Feb. 3, 1998 with the American Type Culture Collection (ATCC), 12301 Parklawn Drive, Rockville, Md. 20852, U.S.A. under the provisions of the Budapest Treaty for the International Recognition of the Deposit of Microorganism for the Purposes of Patent Procedure. The hybridoma was accorded ATCC Accession Number HB-12467. The B152 antibody was developed to C5 hCG as disclosed in WO 98/10282, Prenatal Screening for Down's Syndrome Using Hyperglycosylated Gonadotropin; Cole et al., (1998) Hyperglycosylated hCG, a Potential Alternative to hCG in Down Syndrome Screening, Prenatal Diagnosis, 18:926-933; Cole et al., (1999) Hyperglycosylated Human Chorionic Gonadotropin (Invasive Trophoblast Antigen) Immunoassay: A New Basis for Gestational Down Syndrome Screening, 45:2109-2119. Hybridomas producing the B152 monoclonal antibody were obtained from Columbia University.

As used herein, a “detection antibody” is defined as an antibody, preferably a monoclonal antibody, that specifically binds an antigen at a binding site or epitope distinct from that of the capture antibody. As is understood in the art, depending on the amount of cross-reactivity that is desired for related antigens, the specificity of the detection antibody may vary.

In certain embodiments of the invention, the detection antibody is a monoclonal antibody that recognizes the beta subunit of ITA. One example is a monoclonal antibody designated B207. Monoclonal antibody B207 was generated to the beta subunit of hCG, but is cross reactive with the beta subunit of ITA. The hybridoma producing the B207 monoclonal antibody was deposited with the American Type Culture Collection (ATCC), 12301 Parklawn Drive, Rockville, Md. 20852, U.S.A. under the provisions of the Budapest Treaty for the International Recognition of the Deposit of Microorganism for the Purposes of Patent Procedure. The hybridoma was accorded ATCC Accession Number PTA 1626. The B207 mAb was developed and described in Krichevsky et al., (1994) The Development of a Panel of Monoclonal Antibodies to Human Luteinizing Hormone and its Application to Immunological Mapping and Two-Site Assays, Endocrine, 2:511-520; WO 99/41584, Methods for Predicting Pregnancy Outcome in a Subject by hCG Assay; and WO 00/70094, Methods for Predicting Pregnancy Outcome in a Subject by hCG Assay; O'Connor et al., (1998) Differential Urinary Gonadotrophin Profiles in Early Pregnancy and Early Pregnancy Loss, Prenatal Diagnosis, 18:1232-1240. The hybridoma for the B207 mAb was obtained from Columbia University.

In reference to the disclosure herein, one combination of antibodies is a capture antibody that specifically binds ITA, and a detection antibody that specifically or non-specifically binds ITA. In particular, the capture antibody may be generated from immunization with hyperglycosylated forms of hCG or fragments thereof. The detection antibody may be generated using either hyperglycosylated forms of hCG, normal glycosylated forms of hCG, or fragments thereof. With respect to one embodiment of the invention, the B152 mAb is the capture antibody, and the B207 mAb is the detection antibody. However, in other embodiments of the invention, it is possible to utilize the B207 mAb as the capture antibody, and the B152 mAb as the detection antibody.

A “label” is a composition detectable by spectroscopic, photochemical, biochemical, immunochemical, or chemical means. For example, useful labels include fluorescent dyes, chemilurninescent compounds, radioisotopes, electron-dense reagents, enzymes, colored particles, biotin, or dioxigenin. A label often generates a measurable signal, such as radioactivity, fluorescent light, color, or enzyme activity, which can be used to quantitate the amount of bound label.

Examples of chemiluminescent compounds include luciferin, a luminol derivative, pyrogallol, isoluminol, aequorin, cyclic arylhydrazides, dioxetanes, rhodium chelates (electrochemiluminescent), oxalate esters, thermochemiluminescent labels, acridinium and the like. These labels may be attached to a protein, for example an anti-ITA antibody, using techniques well known in the art. (See U.S. Pat. No. 5,284,952, the disclosure of which is incorporated in its entirety herein by reference.) In one embodiment, a detection antibody, such as B207, may be labeled with an acridinium by employing the methods found in U.S. Pat. Nos. 5,284,952, 5,110,932, and 5,338,847, the disclosures of which are incorporated in their entirety herein by reference.

Examples of the fluorescent material to be used for labeling include fluorescein, fluorescamine, fluorescein isothiocyanate, umbelliferone, rhodamine, Texas red dyes, pthalocyanines, coumarin, squaraine, anthracene, erythrosine, europium chelates and the like.

Examples of radioactive isotopes to be used for labeling include ¹⁴C, ³H, ³²P, ¹⁸F or ¹²⁵I.

Exemplary enzymes which have been developed and can be used in assays of the invention are those described in U.S. Pat. Nos. 3,654,090; 3,791,932; 3,839,153; 3,850,752; 3,817,837; 3,879,262; Journal of Immunological Methods 1:247 (1972); and the Journal of Immunology 109:129 (1972), the disclosures of which are incorporated in their entirety herein by reference. Other examples of enzymes include, but are not limited to, alkaline phosphatase, beta galactosidase, horseradish peroxidase, gluconidase, phosphatase, peptidase, alkaline phosphatase and the like. Co-enzymes useful in this invention include molecules and/or proteins which facilitate an enzyme to catalyze a reactant to produce a detectable product, for example light. A co-enzyme may include, without limitation, FAD and NAD.

Examples of colored particles include colloidal gold, or blue latex.

Other labels may include a non-active precursor of a spectrophotometrically-active substance (British Pat. No. 1,392,403 and French Pat. No. 2,201,299, which patents correspond to U.S. Pat. No. 3,880,934) and electron spin resonance moieties (U.S. Pat. No. 3,850,578).

As described herein, certain parameters of the assays used to practice the methods of the invention are determined prior to practicing the methods. For example, the components of the solutions and their concentrations (e.g., the concentrations of capture and detection is antibodies); the experimental conditions of the assays, such as buffer solution, pH, ionic strength, temperature, incubation times, solid phase support; the coupling chemistry between the support and the various antibodies, and the coupling chemistry between the detection antibody and the label, may be predetermined by conducting conventional experiments to optimize the methods of the invention.

The present invention is, in part, based upon the discovery that the occurrence of Down's syndrome in a fetus may be detected with increased sensitivity and accuracy by measuring a pregnant woman's level of ITA with a combination of antibodies, as disclosed herein. The methods of the invention also enable one to determine an increased risk of a woman carrying a Down's syndrome fetus.

The methods for detecting Down's syndrome disclosed herein comprise screening a biological sample from a pregnant woman for ITA, alone or in combination with other antigens or biological markers, and comparing the amount of ITA to a standard. The sample is screened for ITA, or other biological markers, by contacting the sample with an antibody the specifically binds ITA, or the other biological markers.

Biological samples useful for practicing the methods of the invention include, but are not limited to, whole blood, serum, plasma, urine, and amniotic fluid. In addition, the samples may include tissue samples, such as, for example, tissue from the placenta of a pregnant woman. In certain embodiments of the invention, the biological sample is serum.

Samples may be obtained from the pregnant woman during the first trimester of pregnancy (from about 1 to about 13 weeks) or during the second trimester of pregnancy (from about 13 to about 27 weeks). Samples may be obtained from pregnant women by any conventional method known to those skilled in the art. For example, serum samples may be obtained by withdrawing a volume of blood from the pregnant woman using conventional intravenous techniques. Amniotic samples can be obtained by withdrawing amniotic fluid from pregnant women using a needle and syringe. Urine samples can be obtained from the pregnant woman. The biological samples, such as the serum sample, can be stored before exposing the sample to an assay used to practice the methods of the invention. In practicing the methods of the invention, serum samples may be stable for about three days at room temperature (about 21 degrees C), for about seven days at about 4 degrees C; and for about three years at about −60 degrees C.

Screening the biological sample for ITA is performed by exposing the sample to antibodies that specifically bind ITA.

In one embodiment of the invention, “sandwich” type immunoassays are utilized to measure ITA in a sample. The methods of the invention may utilize a capture antibody that specifically binds ITA. The capture antibody may be coupled to a solid substrate or solid phase. Examples of suitable substrates include, but are not limited to, nylon or nitrocellulose membranes or wells of microtiter plates or cuvettes. In one embodiment of the invention, the capture antibodies are coupled to paramagnetic particles in cuvettes. For example, biotin-coupled capture antibodies can couple to streptavidin coated paramagnetic particles via the well known avidin-biotin binding reaction. Other methods of coupling the capture antibody to the solid phase of the assays are known to those skilled in the art. In one embodiment of the invention, the capture antibody is designated B152. The B152 monoclonal antibody specifically binds ITA, as described in WO 98/10282, Prenatal Screening for Down's Syndrome Using Hyperglycosylated Gonadotropin; WO 99/41584, Methods for Predicting Pregnancy Outcome in a Subject by hCG Assay; WO 00/70094, Methods for Predicting Pregnancy Outcome in a Subject by hCG Assay; O'Connor et al., (1998) Differential Urinary Gonadotrophin Profiles in Early Pregnancy and Early Pregnancy Loss, Prenatal Diagnosis, 18:1232-1240; Cole et al., (1999) Hyperglycosylated Human Chorionic Gonadotropin (Invasive Trophoblast Antigen) Immunoassay: A New Basis for Gestational Down Syndrome Screening, Clinical Chemistry, 45:2109-2119; Cole et al., (1999) Urinary Screening Tests for Fetal Down Syndrome: II. Hyperglycosylated hCG, Prenatal Diagnosis, 19:351-359; and Shahabi et al., (1999) Serum Hyperglycosylated hCG: a Potential Screening Test for Fetal Down Syndrome, Prenatal Diagnosis, 19:488-490.

In practicing the sandwich immunoassay, ITA may also be exposed to a detection antibody that is coupled to a detectable label. Examples of suitable labels are described above, one example of a label is acridinium. Methods of coupling labels to antibodies are well known in the art. For example, acridinium, as a “sulfonyl chloride ester” can be crosslinked to the detection antibody by the reaction of the lysly moiety of the epsilon amino group of lysine in proteins, such as antibodies, to the acridinium ester. The reaction products may then be separated by size exclusion chromatography on Sepharose beads. One detection antibody is designated B207. B207 was developed to the hCG β fragment as described in Krichevsky et al., (1994) The Development of a Panel of Monoclonal Antibodies to Human Luteinizing Hormone and its Application to Immunological Mapping and Two-Site Assays, Endocrine, 2:511-520.

In certain embodiments of the invention, the sandwich immunoassays may be chemiluminescent immunoassays. The range of sensitivity of ITA concentration of the assays disclosed herein may be from about 1 to about 300 ng/mL, but sensitivities of about 0.1 ng/mL are also encompassed. The chemiluminescent assays provide increased sensitivity to current assays used to detect Down's syndrome. Cross-reactivity of the capture antibodies used in the methods of the invention with hCG, β-hCG, and nicked hCG may be less than about 4.5%.

The monoclonal antibodies disclosed herein can also be produced using conventional methods known in the art. See, for example, Kohler and Milstein,(1975) Nature, 256:495-97; or Sambrook et al. (2001) Molecular Cloning: A Laboratory Manual, 3rd edition, Cold Spring Harbor Laboratory Press. Briefly, animals, such as mice, are injected with ITA, or fragments thereof, that may be coupled to a carrier protein. The animals are boosted with one or more ITA injections, and are hyperimmunized by an intravenous (IV) booster about three days before fusion. Spleen cells from the mice are isolated and are fused by standard methods to mycloma cells. Hybridomas are selected in standard hypoxanthine/aminopterin/thymine (HAT) mediunm according to standard methods. Hybridomas secreting antibodies which recognize different epitopes of ITA are identified, cultured, and subdloned using standard immunological techniques. The antibodies are screened for specific binding to ITA.

Although one embodiment of the invention employs sandwich immunoassays to practice the methods of the invention, other immunoassays, such as radioimmunoassays, or competitive immunoassays, may be used. The parameters and components of the assays are determined and optimized as is well known to those skilled in the art such that the assays provide measurement of ITA levels in the biological samples being assayed. In addition, although certain embodiments of the invention utilize antibodies as the agents capturing the ITA, ITA may be captured in the assays of the invention using other chemical agents or molecules that are not antibodies. For example, such an agent may recognize carbohydrate profiles of ITA, and thereby bind the ITA to a solid phase in a similar manner as the capture antibodies described herein.

After screening the sample for ITA, the measured value of ITA is compared to a standard. In one embodiment of the invention, the standard may be obtained from a population of pregnant women who had normal pregnancies. In other words, the pregnant women of the population had normal fetuses, or in particular, the fetuses did not have Down's syndrome. The ITA can be measured as a concentration (e.g., ng/mL) or as a multiple of medians (MoM). For example, the ITA measured in a sample can be compared to the 50^{th} percentile of the ITA values for a population of normal pregnant women. If the ITA value of the sample is greater than the 50^{th} percentile, there would be a significant chance that the fetus of the pregnant mother has Down's syndrome. Similarly, the ITA value could be compared to the 95^{th} percentile of ITA values for a population of normal pregnant women. If the ITA value from the sample was greater than the 95^{th} percentile, the relative likelihood would be even greater that the fetus of the pregnant woman being tested had Down's syndrome. Thus, an amount of ITA in the sample that is higher than a standard ITA amount may indicate the presence of Down's syndrome in the fetus of the pregnant woman.

As discussed herein, when ITA is measured and combined with the pregnant woman's age, the accuracy of measurement is about 80%, for example about 79%, with a 5% false positive rate. The accuracy of detecting Down's syndrome may improve when the methods of the invention are practiced with one or more additional markers. Some examples of markers that may be employed in conjunction with an ITA component in practicing the methods of the invention, include, but are not limited to, free hCG, beta-subunit hCG, beta-core hCG, unconjugated estriol (UE3), alpha-fetoprotein (AFP), leptin, prorenin, renin, DHEA-S, leukocyte acid phosphatase, inhibin, pregnancy associated plasma protein A (PAPP-A), AFP-L3, P43, superoxide dismutase (SOD), proMBP, fetal DNA, insulin-like growth factor binding proteins 3 (IGFBP3), CA 125, placental lactogen, Hp2FF, serum sialytransferase, s100b protein, schwangers chafts protein 1 (SPI), activin A/follistatin, fetal antigen (FA-2) and placental alkaline phosphatase (PALP). The methods may also be practiced by using ultrasound screens in conjunction with the ITA screens.

For example, when ITA is combined with AFP, and the woman's age, the detection rate may be about 85% with a 5% false positive rate. When ITA is combined with UE3, inhibin, and woman's age, the detection rate may be about 90% with a 5% false positive rate. When ITA is combined with UE3, hCG, inhibin, and AFP, the detection rate may be about 95% with a 5% false positive rate.

In addition, the methods may be practiced by combining results of pregnancy screens obtained separately at different times of pregnancy. For example, the results of an ultrasound screen performed during the first trimester of pregnancy may be combined with the results of an ITA screen performed later during the first trimester, or performed during the second trimester of pregnancy.

In certain embodiments of the invention, the methods are automated as much as practical in order to improve replicability of the results and reduce the time and costs required to conduct the assays. Automated assays used to practice the methods of the invention permit users to conduct at least about 80 tests per hour.

One may also use any conventional, non-automated, assay device to practice the methods of the invention. For example, a conventional microtiter plate can be used to store the various solutions used in performing the assay. The device should permit the biological sample to be exposed to a combination of antibodies. The antibodies may recognize different epitopes of the antigen being assayed. The device should also cause the bound antigen to be retained to a substrate as solutions are added and removed during the assay.

By way of example, and not by way of limitation, wells of a microtiter plate can be coated with streptavidin coated magnetic particles, as described herein. A solution containing biotin coupled capture antibodies (e.g., biotin coupled B152 mAb) is added to the well to enable the coupling of the capture antibodies to the magnetic particles. A concentration of capture antibody is empirically selected (based on expected antigen concentrations) as discussed herein, to permit binding of all, or essentially all, of the ITA that is available in the sample. In that regard, typical ITA concentrations in biological samples are in the nanogram to low microgram range (e.g. 1 ng/ml -5 μg/ml) so that the capture antibody concentrations are in the low to high microgram range (e.g. 1-100 μg/ml). The sample is added to the well. If the sample contains ITA, ITA will bind to the capture antibodies. The well is exposed to a magnetic field to immobilize the magnetic particles. The solution is removed from the well; but ITA will not be removed because it is bound to the antibodies that are bound to the magnetic particles that are immobilized in the well of the microtiter plate. A solution containing the detection antibody coupled to a label (e.g., acridinium labeled B207 mAb) is added to the well containing the bound ITA. As indicated elsewhere herein, the concentration of the detection antibody is preferably selected so that all, or essentially all, of the ITA molecules are bound by the detection antibody. Thus, the detection antibody can be provided at concentrations at least an order of magnitude greater than the expected concentration of the ITA. For example, if ITA has an expected concentration of 100 ng/ml, the detection antibody concentration can be 1000 ng/ml (1 μg/ml). After a sufficient amount of time (from about 10 minutes to about 8 or more hours), determined and optimized empirically as described herein, the well is again exposed to a magnetic field, the solution is then removed, and the sample is washed. The amount of label remaining in the well is then measured (e.g., by a luminometer). The measured values can be quantitative or qualitative. Quantitative results are usually preferred. The measured values may then be compared to a standard.

The following examples are presented to illustrate assays and methods used for the detection of Down's syndrome in fetuses of pregnant women. The methodology and results may vary depending on the parameters of the assays being used, as well as the antigens being screened. The examples are not intended in any way to otherwise limit the scope of the invention.

### EXAMPLES

### Example 1

Assays for Measuring ITA

The below described methods of the invention were practiced with the Nichols Advantage® immunoassay system (Nichols Institute Diagnostics (NID), San Juan Capistrano, Calif.).

A series of solutions were provided and stored in individual vials or containers, as described herein. An assay buffer solution comprises 4% protease-free bovine serum albumin (BSA) in 0.5 M phosphate buffer saline (PBS; pH 7.6). A capture antibody solution comprises 4.2 μg/mL (or 0.42 μg/test) of biotin-coupled capture antibody (B152), 0.5% protease free BSA in 0.5 M PBS, 6% normal mouse serum, and 0.1% mouse gamma globulin at a pH of 7.4. A magnetic particle solution comprises 4 mg/mnL of steptavidin coated magnetic particles (M-270; Dynal Biotech, Inc., Lake Success, N.Y.) in normal mouse serum. A detection antibody solution comprises about 0.1 μg/test of an acridinium ester-labeled detection antibody (B207), 0.4% BSA in 0.1 M PBS at a pH of 6.0. A wash solution comprises a detergent, such as Tween®, in PBS with 0.1% sodium azide as a preservative.

The assay was performed by adding 15 μL of a sample, 260 μL of the assay buffer, 70 μL of the capture antibody solution, and 25 μL of the magnetic particle solution to a well in a plate or cuvette. The solution was allowed to incubate for 30 minutes at 37 degrees C.

After incubation, the plate was exposed to a magnetic field to immobilize the ITA/capture antibody/magnetic particle complex. The supernatant was removed and the well was washed with the wash solution. After sufficient washing, determined and optimized empirically, the plate was removed from the magnetic field, and 50 μL of the detection antibody solution and 250 μL of the normal mouse serum was added to the well. The solution incubated for about 10 minutes at 37 degrees C. Subsequently, the plate was again exposed to a magnetic field to immobilize the detection antibody/ITA/capture antibody/magnetic particle complex. The supernatant was removed and the well was washed. An acid solution comprising hydrogen peroxide in a diluted acid, such as HCl, and a base solution comprising diluted sodium hydroxide were then added to the well to trigger the signal of the acridinium ester. The amount of detected signal was then measured in a luminometer, and the data were recorded. If the detected signal exceeded the sensitivity range of the assay, the sample was diluted with a diluent comprising 0.1% protease free BSA in 0.5 M PBS at pH of 7.4.

The foregoing assay permits about 80 tests per hour to be conducted.

### Example 2

Detection of Down's syndrome during the second trimester of pregnancy.

Serum was obtained from pregnant women during their first or second trimester of pregnancy. Some of the samples had been stored at −60 degrees C. for about 3 years.

The methods were practiced as described in Example 1 using serum as the biological sample.

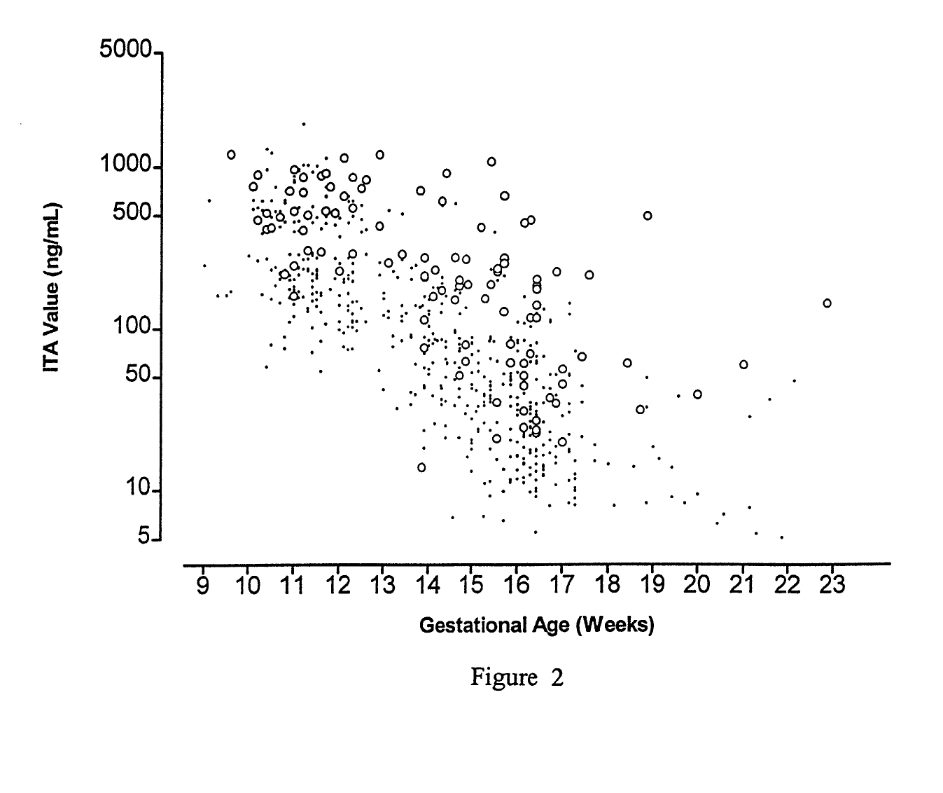

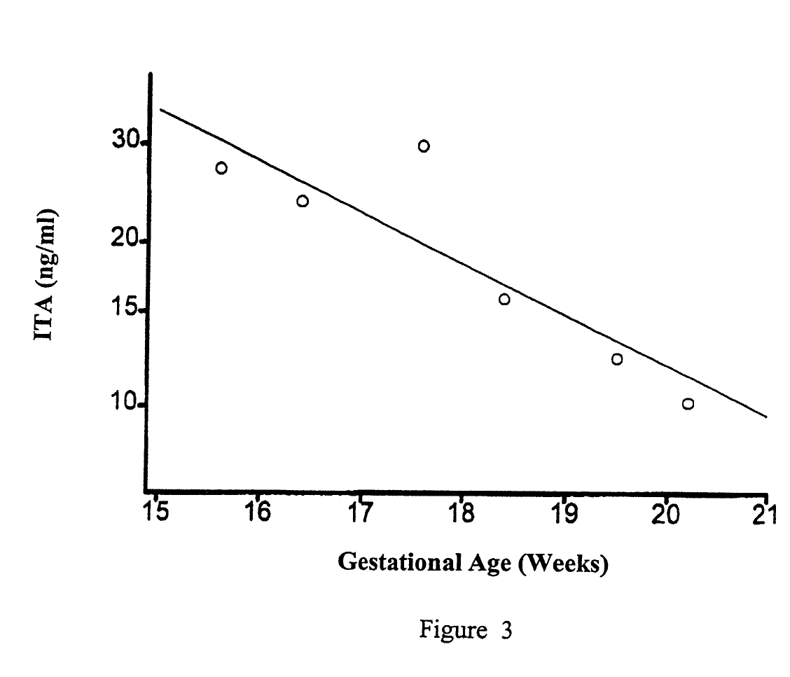

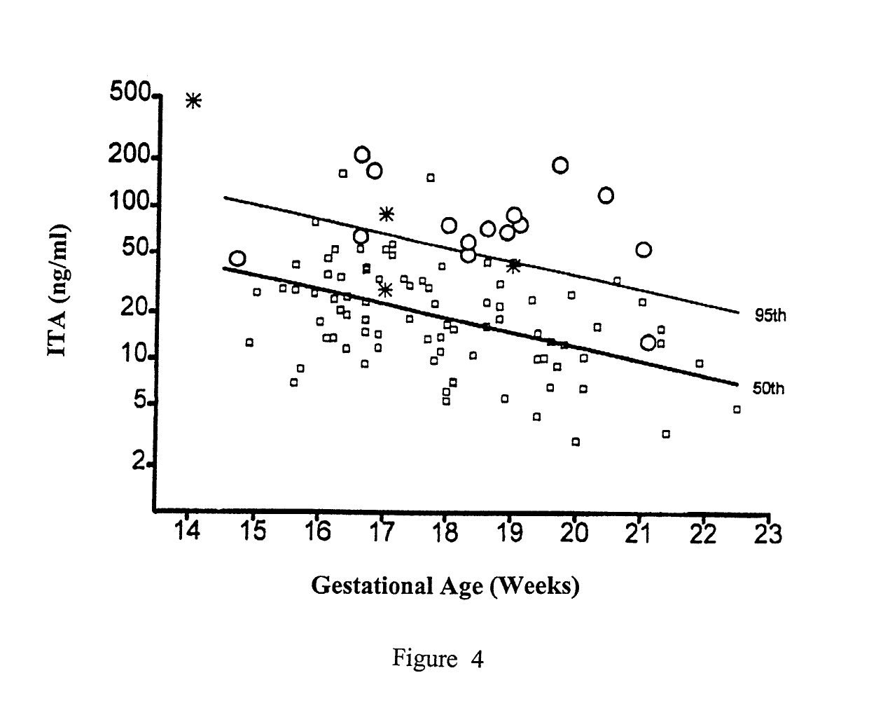

Serum ITA values in control pregnancies decreased by about 19% per week during the second trimester (FIGS. 2 and 3). Smoothed median values at 15 and 20 weeks were about 31.0 ng/mL and about 10.7 ng/mL, respectively. When compared to serum ITA data obtained from women with normal pregnancies, differences among the data were observed (FIG. 4). The measured ITA values from serum of women who had fetuses with Down's syndrome (including values from serum that was frozen) were greater than the 50^{th} percentile of the ITA values for normal pregnancies. In addition, a significant proportion of the ITA values from the Down's syndrome pregnancies exceeded the 95^{th} percentile of the ITA values for normal pregnancies.

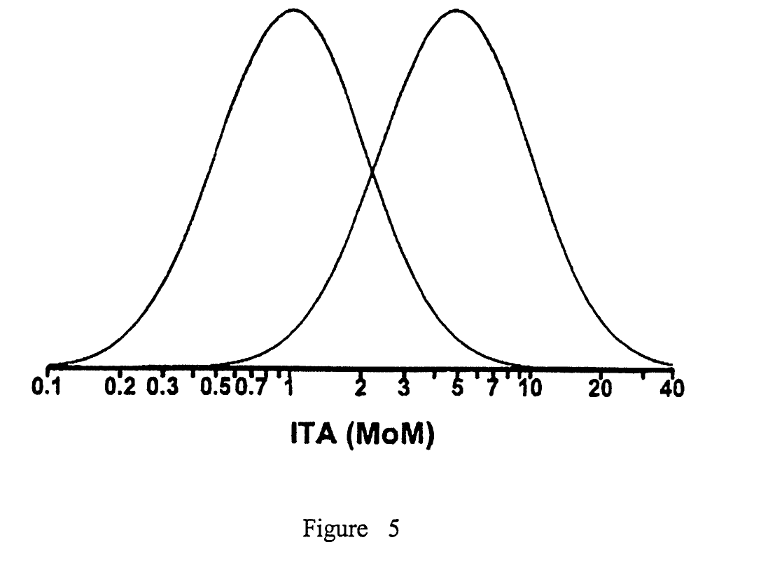

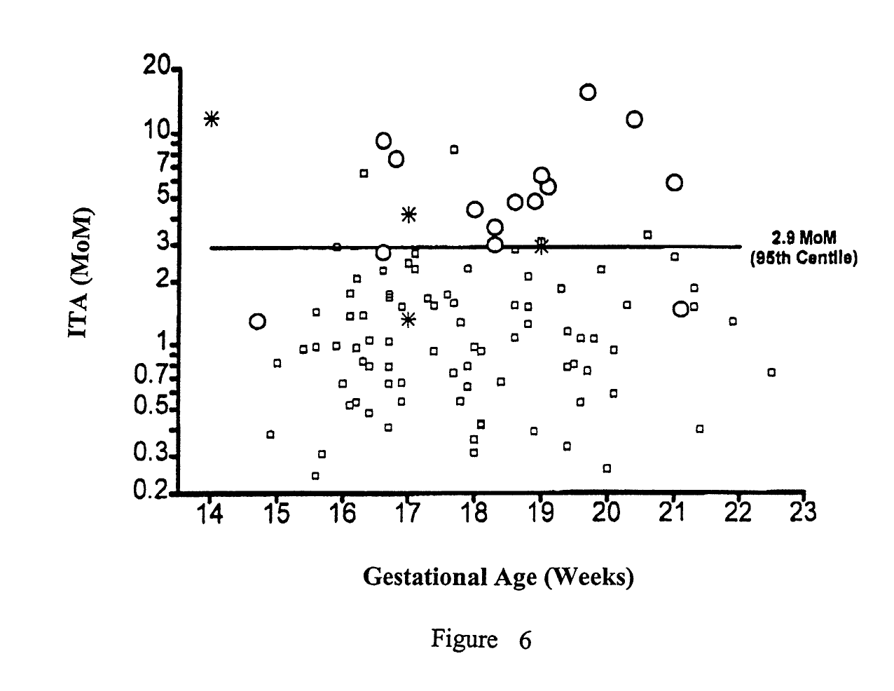

All results were converted to multiples of the gestational age-specific median (MoM). The normal pregnancies had a median ITA level of 1.02 MoM (FIG. 4). The Down's syndrome pregnancies had a median ITA level of 4.99 MoM (range 1.45 to 15.3 MoM; FIGS. 4 and 5). Overall, about 72% of the Down's syndrome cases were above the 95^{th} percentile (2.9 MoM; FIGS. 5 and 6).

When combined with maternal age, detection increased to 79% at a 5% false positive rate. Detection decreased to 72% when the false positive rate is reduced to 3%. Adding α-fetoprotein measurements further increased the detection rate to 87%. For comparison, the most effective combination of markers currently in use (AFP, uE3, hCG, and dimeric inhibin A) can detect 77% of Down's syndrome cases at a 5% false positive rate. In a multivariant analysis using serum ITA, hCG, AFP, uE3, and inhibin-A, the sensitivity was 96% with a 5% false positive rate, 92% with a 3% false positive rate, or 85% with a 1% false positive rate.

Examples of detection rates for various combinations of markers and false positive rates are set forth in Table I below:

TABLE I

| | False Positive Rate | | |
|---|---|---|---|
| Age and Marker(s) | 5% | 3% | 1% |
| ITA | 79% | 72% | 56% |
| ITA & AFP | 87% | 77% | 63% |
| ITA, UE3, hCG, & AFP | 83% | 79% | 63% |
| ITA, UE3, Inhibin | 89% | 82% | 66% |
| ITA, UE3, hCG, Inhibin, & AFP | 96% | 92% | 85% |

Based on these results, second trimester serum ITA levels are about five times higher in Down's syndrome pregnancies than in unaffected pregnancies.

### Example 3

Detection of Down's syndrome during the first trimester of pregnancy

Urine samples were obtained from pregnant women during their first trimester of pregnancy. The urine samples were analyzed for ITA, β-core fragment, and hCG, using the method of Example 1. The urine samples were normalized for creatinine. Serum values for total hCG, pregnancy associated plasma protein A, and free beta hCG for these pregnancies were used for multivariate analyses.

Univariately, the median urine ITA levels in the first trimester was about 3.16 MoM as compared to 2.52 MoM for urine hCG, and 1.50 MoM for urine β-core fragment. The observed detection rate for urine ITA at a false positive rate of 5% was 25%. The observed detection rate for urine ITA at a false positive rate of 10% was 53%. These results were somewhat similar to urine hCG (24% at a 5% false positive rate; and 53% at a 10% false positive rate). The results of urine ITA were better than urine β-core fragment (6% at a 5% false positive rate; and 24% at a 10% false positive rate).

Multivariately, the most powerful combination of 3 analytes was urine ITA with serum pregnancy associated plasma protein, and serum free β-hCG yielding a detection rate of about 74% with a false positive rate of 3%, and a detection rate of about 81% with a false positive rate of 5%. The detection rate is reduced from about 81% to about 74% when urine ITA is replaced by urine hCG.

Thus, urinary ITA combined with serum free β-hCG, and serum pregnancy associated plasma protein provides a first trimester screening performance comparable to that obtained by second trimester screening with four analytes (i.e., AFP, uE3, hCG, and dimeric inhibin A).

### Example 4

In another embodiment, an assay may be conducted as follows. In brief, a 96-well microtiter plate (Nunc Immulon-1; Fisher Scientific) is coated with capture antibody (0.2 mL per well of a solution containing 2.5 mg/L B152 antibody in 0.25 mol/L NaHCO₃ and 0.1 mol/L NaCl) by incubation for 16-24 hours at 4 degrees C. Plates are then washed three times with water and blotted dry, and wells are blocked with phosphate-buffered saline, pH 7.4 (Life Technologies), containing 10 g/L bovine serum albumin and 0.4 g/L sodium azide (both from Sigma). After incubation for 1 hour at ambient temperature, plates are again washed three times with water, blotted dry, and used for the assay. The total assay volume is 0.2 mL determined as 0.1 mL of sample or calibrator and 0.1 mL of phosphate-buffered saline containing 1 g/L bovine serum albumin and 0.4 g/L sodium azide. C5 hCG (100% hexasaccharide-type O-linked oligosaccharides), the irnmunogen for antibody B 152, that has been calibrated by amino acid analysis is used as the calibrator. C5 hCG at concentrations of 0, 6, 12, and 24 μg/L is added to quadruplicate wells of the plate. Biological samples (urine or serum) are added at two- and five-fold dilutions. Buffer is added, and the plates are incubated for 4 hours at ambient temperature on an orbital plate shaker. Plates are again washed three times with water and blotted dry. Tracer antibody [0.2 mL of peroxidase-labeled B207 mAb, 1:5000 titer in Tris, pH 7.3 (Sigma) containing 1 g/L bovine serum albumin and 1.9 g/L CaCl₂·2H₂O] is added to each well. After an additional 2 hour incubation at ambient temperature on a plate shaker, plates are again washed three times with water and blotted dry. Finally, 0.2 mL of substrate [3,3′,5,5′-tetramethylbenzidine (TMB) reagent (Catalog No. T8665; Sigma) diluted 1:1 with water] is added to each well. After a 15 minute incubation at ambient temperature, the reaction is stopped by the addition of 0.05 mL of 2 mol/L HCl. The plates are read on a microtiter plate reader at 450 nm, and the calibrators are plotted. The points best fit a cubic function, which is used to calculate sample values. Plates include a quality control.

### Example 5

Assays for Measuring Creatinine

Results of urine assays disclosed herein may be normalized to spot urine creatinine concentrations to adjust for variations in urine concentration. Creatinine concentration may be determined using a commercial kit, Catalog No. 555A (Sigma), and a nicrotiter plate adaptation of the protocol. Calibrators (0, 2.5, 1.5, 0.5, and 0.2 g/L creatinine) and urine samples (0.053 mL per well, in triplicate) are added to a 96-well microtiter plate. Alkaline picrate reagent is prepared fresh (5 parts of solution plus 1 part sodium hydroxide) and added (250 mL) to the wells. The plate is incubated 15 minutes at ambient temperature. The absorbance is measured at 492 nm by a plate reader, and the calibrators are plotted. The points best fit a cubic equation, which is used to calculate sample concentrations (g/L) (Cole et al., (1999) Clinical Chemistry, 45:2109-2119).

Alternatively, urine creatinine may be measured with a standard Jaffe reaction procedure. Concentration of an analyte is divided by the creatinine concentration to obtain the normalized analyte concentration (Spencer, (1986) Annals of Clinical Biochemistry, 23:1-25).

### Example 6

Assays for Measuring β-core fragment

β-core fragment concentrations may be determined by a method similar to that for the ITA assay. The assay may utilize an antibody designated B210 (obtained from Columbia University, New York, N.Y., U.S.A.), and a different calibrator (P13 β-core fragment). The β-core fragment assay detects hCG β-core fragment. Although this assay has 100% activity with the hLH β-core fragment calibrator, it has no measurable activity with free hCG or any of the intact-hCG calibrators (Cole et al., (1999) Clinical Chemistry, 45:2109-2119).

Alternatively, β-core fragment levels may be determined by the B210 assay, as described previously (Cole et al., (1994) Journal of Clinical Endocrinology and Metabolism, 78:497-499; Isozaki et al., (1997) Prenatal Diagnosis, 17:407-413). This is a two-step sandwich assay. Briefly, microtiter plates are coated with monoclonal antibody B210 (obtained from Columbia University, New York, N.Y., U.S.A.), urine samples are added and β-core fragment extracted. Plates are washed and peroxidase-labeled hCG β-subunit antibody (Bios Specific, Emmerville, Calif., U.S.A.) is added to quantitate bound β-core fragment. After a further wash, substrate is added and peroxidase enzyme activity is measured spectrometrically. Urine samples are diluted with phosphate-buffered saline containing 0.1 percent (w/v) bovine serum albumin (normal dilution buffer) for this assay. Samples are assayed at one or more dilutions, as needed (between 50×and 10,000×). The B210 assay is standardized with pure β-core fragment, purified from pregnancy urine and calibrated by amino acid analysis. The B210 assay detects only β-core fragment, with less than 0.1 percent cross-reactivity with free β-subunit and hCG. Plates include a high- and low-quality control.

### Example 7

Assays for Measuring Free hCG

Urine samples may be diluted 1 in 5 with zero diluent prior to analysis (Spencer et al., (1997) Prenatal Diagnosis, 17:525-538). The following protocol is an adaptation of measuring serum free hCG, and only difference is diluting the urine sample 1 in 5. The assay of free β-hCG may be carried out with a solid-phase two-site immunoradiometric assay (ELSA-FbHCG; CIS (UK) Ltd., High Wycombe, Bucks., U.K.) in which the monoclonal antibodies used are raised against sterically remote epitopes on the β-hCG molecule. The cross-reactivity of the antibodies with free α-subunit and with intact hCG is less than about 0.01%. The assay involves a 1-h incubation at room temperature of 100 mL of sample and 200 mL of assay buffer in the coated ELSA tube; this is followed by a washing step, a further 2 hour room temperature incubation with 300 mL of labeled second antibody, and a final washing step. The bound radioactivity is counted in an NE 1600 multihead gamma-counter (NE Technology Ltd., Reading, U.K.) and the counts are processed by using the WHO mass action curve-fitting routine (Edwards PR, Ekins RP. Mass action model-based mircroprocessor program for RIA data processing. In: Hunter WM, Corrie JET, eds. Immunoassays for clinical chemistry, 2nd ed. Edinburgh: Churchill Livingstone, 1983: 640-52) (Spencer, (1991) Clinical Chemistry, 37:809-814).

Alternatively, the NID Laboratories free β (i.e. free hCG) assay can be used. Although this assay is designed to detect serum free hCG (Macri et al., (1993) Annals of Clinical Biochemistry, 30:94-98), diluting the urine sample as above may work with this assay.

The NID Laboratories free β assay is an enzymelinked immunosorbent assay employing monoclonal and affinity purified polyclonal antibodies. Free β used as standard in the NID free β assay is purchased from UCB Bioproducts (Belgium). All incubations are performed at room temperature on a rotator (200 rpm). Briefly, 20 mL of standard, controls and samples in duplicate are incubated with 100 mL of phosphate buffered saline (PBS) in monoclonal coated 96-well microtiter plates for 30 minutes. After a wash procedure, 100 mL of biotinylated polyclonal antibody is incubated in all wells for 30 minutes. After another wash procedure, the plates are incubated with 100 mL of streptavidin-horseradish peroxidase conjugate for 4.5 minutes. After a final wash procedure, plates are incubated with 100 mL of ortho-phenylenediamine solution for 8.0 minutes after which the reaction is stopped with 100 mL of 1 N H₂SO₄ and the absorbance values are read on a microtiter plate reader. Controls and samples are quantitated from the standard curve (Macri et al., (1993) Annals of Clinical Biochemistry, 30:94-98).

### Example 8

Assays for Measuring Total Estriol

Total estriol may be determined by radioimmunoassay, using a kit sold by Diagnostic Products Corporation (Los Angeles, Calif., U.S.A.). The kit utilizes antibody-coated tubes, estriol-releasing enzyme, radioiodine-labeled tracer, and a set of six standards (Catalog No. TKE35). The procedures are those described in the instruction booklet. Urine samples are initially diluted to 1 to 31 for the immunoassay. Further dilutions, 1 to 1, 1 to 10, and 1 to 100, are made as needed (Cole et al., (1999) Prenatal Diagnosis, 19:340-350; Cole et al., (1997) Prenatal Diagnosis, 17:1125-1133).

Alternatively, total estriol may be measured in duplicate using the Johnson and Johnson Estriol (total) II radioimmunoassay (Johnson and Johnson Clinical Diagnostics Ltd., Amersham, U.K.). Urine samples are either analyzed without dilution, or are diluted 1 in 5 in normal female serum.

In another assay, levels of total estriol are determined using a specific fluorescence polarization immunoassay (TDx total estriol), Abbott Laboratories, Abbott Part, Ill., U.S.A.). Because of high levels of estriol in pregnancy urine, samples may be diluted about 1:100 prior to assay in sample diluent provided with the kit. For total estriol, the 284 and 3647 ng/ml controls have inter-assay coefficients of variations (CVs) of 6.2 and 5.3 percent, respectively, and intra-assay CVs of 4.8 and 3.6 per cent, respectively (Kellner et al., (1997) Prenatal Diagnosis, 17:1135-1141).

Various publications and/or references have been cited herein, the contents of which are incorporated herein by reference.

While this invention has been described with respect to various specific examples and embodiments, it is to be understood that the invention is not limited thereto and that it can be variously practiced with the scope of the following claims.

We claim:

1. A method for detecting Down's syndrome in a fetus of a pregnant woman comprising the steps of:
a) contacting a serum sample from a pregnant woman in a chemiluminescent assay with an antibody that specifically binds to ITA; and
b) comparing the amount of ITA in the sample to a standard ITA amount obtained from a population of pregnant women with normal fetuses, wherein a higher amount of ITA in the sample as compared to the standard ITA amount indicates the presence of Down's syndrome in the fetus.

2. The method of claim 1, wherein the antibody is a monoclonal antibody.

3. The method of claim 2, wherein the monoclonal antibody was raised against the hCG beta fragment.

4. The method of claim 3, wherein the monoclonal antibody is designated B207.

5. The method of claim 4, wherein step (a) is a sandwich immunoassay method, and the capture antibody is designated B152, and the detection antibody is designated B207.

6. The method of claim 5, wherein the monoclonal antibody B152 is coupled to biotin, and is bound to streptavidin coated magnetic particles.

7. The method of claim 6, wherein the monoclonal antibody B207 is coupled to an acridinium ester.

8. The method of claim 1, further comprising the step of analyzing at least one additional analyte predictive of an increased risk of a fetus being affected by Down's syndrome, the additional analyte is selected from the group consisting of hCG, beta-subunit hCG, beta-core hCG, unconjugated estriol, α-fetoprotein, leptin, prorenin, renin, DHEA-S, leukocyte acid phosphatase, inhibin A, PAPP-A, AFP-L3, P43, SOD, proMBP, fetal DNA, IGFBP3, CA 125, placental lactogen, Hp2FF, serum sialytransferase, s100b protein, SPI, activin A/follistatin, FA-2, and PALP.

9. The method of claim 1, further comprising the step of analzying the results of an ultrasound screen of the fetus of the pregnant woman.

10. A method for determining an increased risk of a woman carrying a Down's syndrome fetus comprising the steps of:
a) contacting a biological sample from a pregnant woman in a chemiluminescent immunoassay with a monoclonal antibody designated B207 that binds ITA; and
b) comparing the amount of ITA in the sample to a standard ITA amount obtained from a population of pregnant women with normal fetuses, wherein a higher amount of ITA in the sample as compared to the standard ITA amount indicates the presence of Down's syndrome in the fetus.

11. The method of claim 10, wherein step (a) further employs a monoclonal antibody that specifically binds ITA.

12. The method of claim 11, wherein the monoclonal antibody is designated B152.

13. The method of claim 12, wherein step (a) is a chemiluminescent sandwich immunoassay, and wherein the B207 mAb is a detection antibody and the B152 mAb is a capture antibody.

14. The method of claim 10, wherein the amount of ITA in the sample as determined in step (b) is above the 50^{th} percentile of ITA found in a population of women with normal pregnancies.

15. The method of claim 10, wherein the amount of ITA in the sample as determined in step (b) is above the 95^{th} percentile of ITA found in a population of women with normal pregnancies.

16. The method of claim 10, wherein the sample is obtained during the second trimester of pregnancy.

17. The method of claim 10, wherein the sample is obtained during the first trimester of pregnancy.

18. The method of claim 10, further comprising screening the sample for at least one marker selected from the group consisting of α-fetoprotein, unconjugated estriol, inhibin, and hCG.

19. The method of claim 18, wherein the at least one marker is α-fetoprotein.

20. The method of claim 18, wherein the at least one marker is α-fetoprotein and inhibin.

21. The method of claim 18, wherein the at least one marker is α-fetoprotein, unconjugated estriol, and hCG.

22. The method of claim 18, wherein the at least one marker is unconjugated estriol, and inhibin.

23. The method of claim 18, wherein the at least one marker is unconjugated estriol, hCG, inhibin, and α-fetoprotein.

24. The method of claim 10, wherein the detection rate is greater than about 80%.

25. The method of claim 10, wherein the detection rate is greater than about 85%.

26. The method of claim 10, wherein the detection rate is greater than about 90%.

27. The method of claim 10, wherein the detection rate is greater than about 95%.

28. The method of claim 10, wherein the screening is automated.

29. The method of claim 10, wherein the standard is obtained from samples from women with normal pregnancies.

30. A method for detecting Down's syndrome in a fetus of a pregnant woman comprising the steps of:
a) contacting a sample from a pregnant woman in an automated chemiluminescent immunoassay with a capture monoclonal antibody designated B152 that specifically binds ITA, and a detection monoclonal antibody designated B207; and
b) comparing the amount of ITA in the sample to a standard ITA amount obtained from a population of pregnant women with normal fetuses, wherein a higher amount of ITA in the sample as compared to the standard ITA amount indicates the presence of Down's syndrome in the fetus.

31. The method of claim 30, wherein the sample is a whole blood sample.

32. The method of claim 30, further comprising screening the sample for unconjugated estriol, hCG, inhibin, and α-fetoprotein.

33. A method for detecting Down's syndrome in a fetus of a pregnant woman comprising the steps of:
a) screening a sample from a pregnant woman for antigens, ITA, unconjugated estriol, hCG, inhibin, and α-fetoprotein; and
b) comparing the amount of the antigens in the sample to standard antigen amounts obtained from a population of pregnant women with normal fetuses, thereby detecting Down's syndrome in the fetus.

34. The method of claim 33, wherein the sample is a whole blood sample, a serum sample, or a urine sample.

35. The method of claim 33, wherein the sample obtained in step (a) comprises two samples consisting of a serum sample and a urine sample.
